(19) United States
(12) Patent Application Publication
(10) Pub. No.: US 2025/0135219 A1
(43) Pub. Date: May 1, 2025

(54) ELECTROMAGNETIC WAVE STIMULATION OPTIMIZATION SYSTEM AND METHOD

(71) Applicant: HANKUK UNIVERSITY OF FOREIGN STUDIES RESEARCH & BUSINESS FOUNDATION, Yongin-si (KR)

(72) Inventors: Bongkeun KANG, Daegu (KR); Hyungkyu HUH, Daegu (KR); Seongmin KIM, Daegu (KR); Myung-kyun WOO, Seoul (KR)

(73) Assignee: HANKUK UNIVERSITY OF FOREIGN STUDIES RESEARCH & BUSINESS FOUNDATION, Yongin-si (KR)

(21) Appl. No.: 18/934,289

(22) Filed: Nov. 1, 2024

(30) Foreign Application Priority Data
Nov. 1, 2023 (KR) 10-2023-0148789

(51) Int. Cl. A61N 5/00

(52) U.S. Cl. CPC A61N 5/00

(57) ABSTRACT

The present disclosure relates to electromagnetic wave stimulation optimization system and method. The electromagnetic wave stimulation optimization system includes an input unit configured to receive information on a target person and information on an EM wave; a simulation unit configured to apply the received information on the target person and the received information on the EM wave to a previously stored human phantom to perform a simulation and configured to predict a human body response to EM wave stimulation; and an output unit configure dot output an optimal EM wave stimulation condition from the human body response of the EM wave stimulation for the information on the at least one EM wave.

### BACKGROUND OF THE INVENTION

The present disclosure relates to electromagnetic (EM) wave stimulation optimization system and method, and more specifically, to electromagnetic wave stimulation optimization system and method that simulate a human body's response to a set EM wave stimulation and derive optimal EM wave stimulation conditions based on the simulation result.

EM waves are generated when electric and magnetic fields change over time, and include gamma rays (γ-rays), X-rays, ultraviolet rays, visible light, infrared rays, microwaves, radio waves, and so on. EM waves stimulate a molecular movement of a material in the process of passing through the material, thereby generating heat, and are used to examine and diagnose bones or skeletons or treat a part of the body.

When applying EM waves to a target person for a set period of time (for example, 30 minutes), a change in body temperature or blood flow of a part of the target person's body may occur, causing side effects such as tickling or hot sensations in the target person.

In addition, even when EM waves of the same intensity are applied to a part of the body, effects may appear differently for each target person because target persons have different heights, weights, ages, muscle mass, and body fat mass.

Therefore, technology is needed to derive optimal EM wave stimulation condition based on the information on a target person and the information on a body stimulation part of the target person.

The technology that serves as the background of the present disclosure is described in Korean Patent Publication No. 10-2022-0146772 (published on Nov. 2, 2022).

This work was supported by the Korea Basic Science Institute (National research Facilities and Equipment Center) grant funded by the Korea government (MSIT) (RS-2024-00402891).

### SUMMARY OF THE INVENTION

The present disclosure provides electromagnetic (EM) wave stimulation optimization system and method that simulate a human body's response to a set EM wave stimulation and derive optimal EM wave stimulation conditions based on the simulation result.

According to an aspect of the present disclosure, an electromagnetic wave stimulation optimization system includes an input unit configured to receive information on a target person and information on an EM wave; a simulation unit configured to apply the received information on the target person and the received information on the EM wave to a previously stored human phantom to perform a simulation and configured to predict a human body response to EM wave stimulation; and an output unit configure dot output an optimal EM wave stimulation condition from the human body response of the EM wave stimulation for the information on the at least one EM wave.

The input unit may set a stimulation area and a purpose of the EM wave based on the received information on the target person.

The information on the target person may include an age, a height, a weight, gender, a medical history, a symptom, a stimulation area, and a specific position of the stimulation area of the target person, and the information on the EM wave may include a frequency, an amplitude, a stimulation time, a stimulation pulse, and a stimulation pulse pattern of the EM wave.

The simulation unit may apply the received information on the target person and the received information on the EM wave to a pre-stored human body phantom to perform a human body response simulation based on a Finite-difference time-domain (FDTD) and predict a change in local body temperature of the stimulation area and a change in local blood flow of the stimulation area.

The output unit may match an optimal body temperature change to an optimal blood flow change according to a height, a weight, and a stimulation area of the target person and use of the EM wave, store a matching result in advance, and derive information on an EM wave causing a body temperature change and a blood flow change that are respectively most similar to the change in body temperature and the change in blood flow as information on an optimal EM wave.

According to another aspect of the present disclosure, an EM wave stimulation optimization method includes receiving information on a target person and information on an EM wave; applying the received information on the target person and the received information on the EM wave to a previously stored human phantom to perform a simulation and predicting a human body response to an EM wave stimulation; and outputting an optimal EM wave stimulation condition from the human body response of the EM wave stimulation for the information on the EM wave.

In this way, according to an embodiment of the present disclosure, a guideline may be provided to perform precise treatment by generating and applying an optimal EM wave in consideration of a target person's information.

The optimal EM wave may be generated by performing a simulation using information on a target person and information on an EM wave and by using the information on the optimal EM wave according to the purpose of rehabilitation, healthcare, or exercise prescription.

### BRIEF DESCRIPTION OF THE DRAWINGS

FIG. 1 is a diagram illustrating an electromagnetic wave stimulation optimization system according to an embodiment of the present disclosure.

FIG. 2 is a flowchart illustrating an electromagnetic wave stimulation optimization method according to another embodiment of the present disclosure.

FIG. 3 is a schematic view schematically illustrating an electromagnetic wave stimulation optimization method according to another embodiment of the present disclosure.

FIG. 4 is a view illustrating an example of a human phantom according to another embodiment of the present disclosure.

FIG. 5 is a view illustrating a comparison between a simulation generated through a prediction model according to another embodiment of the present disclosure and a magnetic field measurement result shown by using a magnetic resonance imaging scanner (MRI).

FIG. 6 is a view illustrating a change in temperature of a human body generated through electromagnetic waves in a certain frequency range through a prediction model according to another embodiment of the present disclosure.

### DETAILED DESCRIPTION OF THE INVENTION

Hereinafter, preferred embodiments according to the present disclosure will be described in detail with reference to the attached drawings. In this process, thicknesses of lines and sizes of components illustrated in the drawings may be exaggerated for the sake of clarity and convenience of description.

Throughout the specification, when a portion “includes” a component, the portion may further include other components instead of excluding other components, unless otherwise specifically stated.

In addition, the terms described below may be defined in consideration of functions of the present disclosure and may change depending on the intention or custom of a user or operator. Therefore, definitions of the terms should be made based on the contents throughout the specification.

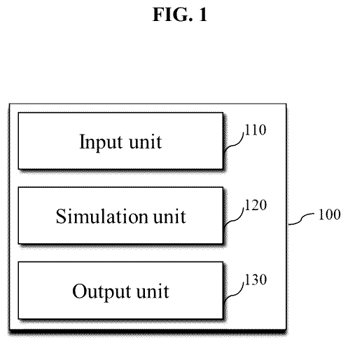

FIG. 1 is a diagram illustrating an electromagnetic wave stimulation optimization system according to an embodiment of the present disclosure.

As illustrated in FIG. 1, an electromagnetic wave stimulation optimization system 100 may include an input unit 110, a simulation unit 120, and an output unit 130.

First, the input unit 110 may receive information on a target person and information on at least one EM wave through a user interface. Here, the information on the target person may include an age, a height, a weight, gender, a stimulation area, and a certain position of the stimulation area of the target person and may further include at least one of a medical history, muscle mass, and body fat mass of the target person as needed, and the information on the EM wave is information on a radio frequency (RF) set to a range of several Hz to several MHz and may include a frequency, an amplitude, a stimulation time, a stimulation pulse, a stimulation pulse pattern (for example, a sine wave, a square wave, an adiabatic pulse, or so on), and so on.

Specifically, the input unit 110 may set a stimulation area and the purpose of an EM wave based on the input information on the target person. Here, the purpose of an EM wave may be any one of inspection, cancer cell destruction, hyperthermia treatment of the stimulation area, rehabilitation, healthcare, or exercise prescription.

Next, the simulation unit 120 may perform a simulation by applying the input information on a target person and the information on an EM wave to a pre-stored human phantom and predict a human body response to the EM wave stimulation. In this case, the human body phantom may include information on pre-stored multiple human tissues according to a height and weight and may indicate human body responses, such as changes in body temperature and blood flow of the stimulation area to the EM wave stimulation.

Specifically, the simulation unit 120 may apply the input information on a target person and the information on at least one EM wave to the pre-stored human body phantom, perform a human body response simulation based on a numerical analysis method, such as a FDTD, a finite element method (FEM) or a method of moments (MOM), and predict changes in local body temperature and local blood flow of the stimulation area.

In addition, the simulation unit 120 may precisely predict a human body response to the EM wave stimulation through the pre-stored human body phantom based on change amounts of body temperature and blood flow of the stimulation area predicted previously for the information on multiple EM wave stimulation.

In addition, the simulation unit 120 may match the predicted changes in body temperature and blood flow of the stimulation area with the information on the target person and the information on the EM wave and transmit the matching result to the output unit 130.

Next, the output unit 130 may output the optimal EM wave stimulation condition from the human body response of the EM wave stimulation for the information on at least one EM wave.

Specifically, the output unit 130 may output the optimal EM wave stimulation condition from the human body response of at least one EM wave stimulation based on the purpose of the EM wave generated by the input unit 110 and the information on the stimulation area and the target person. In this case, the output unit 130 may match the optimal change in body temperature to the optimal change in blood flow according to the height, weight, and stimulation area of the target person, and the purpose of an EM wave and store the matching result in a database (not illustrated).

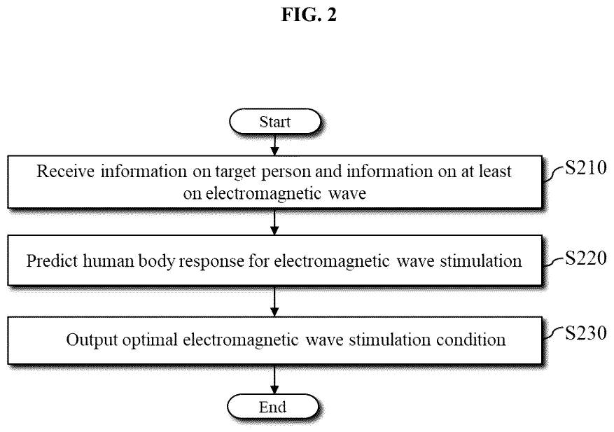

Hereinafter, the EM wave stimulation optimization method will be described in more detail with reference to FIGS. 2 to 6.

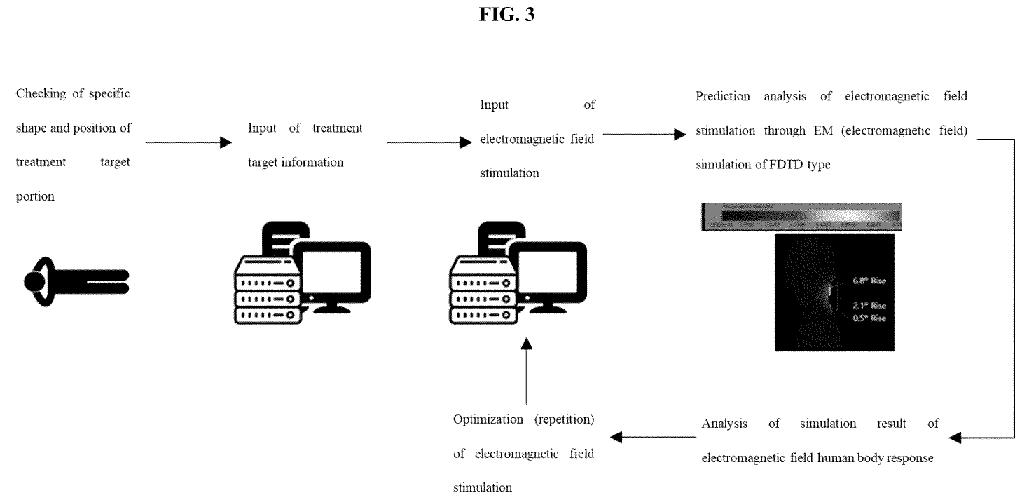

FIG. 2 is a flow chart illustrating an electromagnetic wave stimulation optimization method according to another embodiment of the present disclosure, and FIG. 3 is a schematic view schematically illustrating an electromagnetic wave stimulation optimization method according to another embodiment of the present disclosure.

As illustrated in FIG. 2 and FIG. 3, the input unit 110 may receive information on a target person and information on at least one EM wave (S210). Here, the information on a target person may include an age, a height, a weight, gender, a medical history, a symptom, a stimulation area, and a specific position of the stimulation area of the target person and may further include a specific shape of the stimulation area, muscle mass, and body fat mass as needed, and the information on an EM wave is information on a RF set in a range from several Hz to several MHz and may include a frequency, an amplitude, a stimulation time, a stimulation pulse, a stimulation pulse pattern, and so on.

Specifically, the input unit 110 may set or receive the purpose of an EM wave based on the information on a target person input through the user interface. Here, the purpose of the EM wave may be any one of inspection, cancer cell destruction, hyperthermia treatment of a stimulation area, rehabilitation, healthcare, or exercise prescription based on a medical history, a symptom, and a stimulation area of the target person.

Next, the simulation unit 120 may perform a simulation by applying the input information on the target person and the information on an EM wave to a pre-stored human phantom and predict the human body response to the EM wave stimulation (S220). In this case, the human body phantom may include information on pre-stored multiple human tissues according to a height and weight and may indicate human body responses, such as changes in body temperature and blood flow of the stimulation area to the EM wave stimulation.

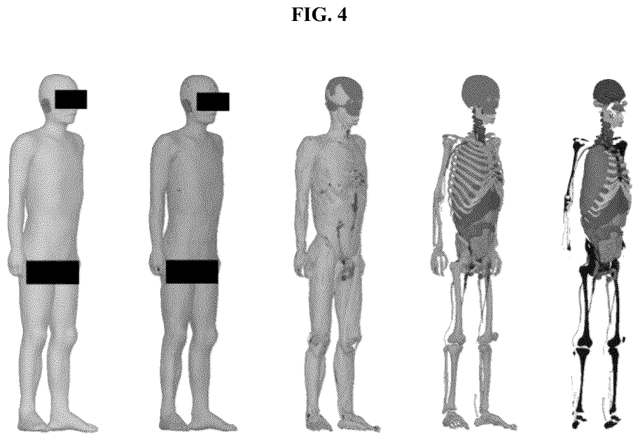

FIG. 4 is a view illustrating an example of a human phantom according to another embodiment of the present disclosure.

As illustrated in FIG. 4, the human phantom may be normalized through a specific shape and position of the stimulation area and the information on a target person and may be stored.

In addition, the simulation unit 120 may predict a change in body temperature and a change in blood flow of the stimulation area by performing at least one simulation according to the information on an EM wave, and when performing two or more simulations, change amounts of body temperature and blood flow of the stimulation area predicted previously may be additionally applied to predict changes in body temperature and blood flow of the stimulation area.

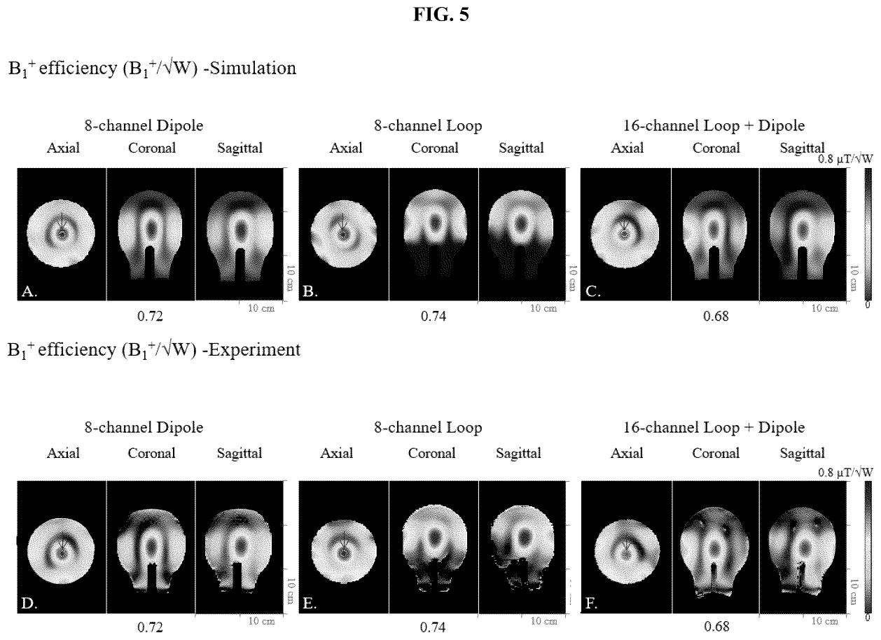

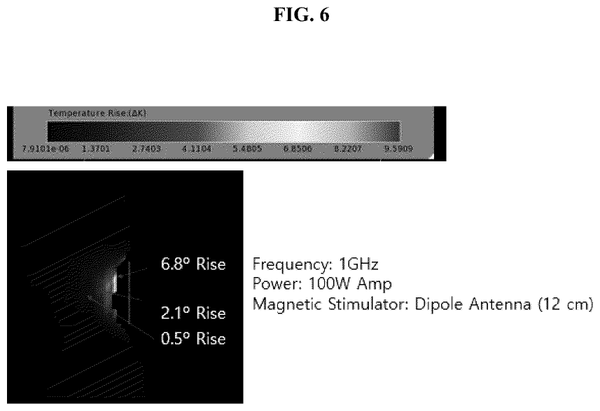

FIG. 5 is a view illustrating a comparison between a simulation generated through a prediction model according to another embodiment of the present disclosure and a magnetic field measurement result shown by using a magnetic resonance imaging scanner (MRI), and FIG. 6 is a view illustrating a change in temperature of a human body generated through electromagnetic waves in a certain frequency range through a prediction model according to another embodiment of the present disclosure.

As illustrated in FIGS. 5 and 6, the simulation unit 120 may generate a simulation result showing changes in body temperature and blood flow of a stimulation area according to an angle at which EM waves are emitted through a prediction model. In this case, a dipole antenna having a frequency of an EM wave of 1 GHz, power of 100 W, and a length of 12 cm is provided, but the present disclosure is not limited thereto.

In addition, the simulation unit 120 may match the predicted changes in body temperature and blood flow of the stimulation area with the information on the target person and the information on the EM wave and transmit the matching result to the output unit 130.

Next, the output unit 130 may output the optimal EM wave stimulation condition from the human body response of the EM wave stimulation for the information on at least one EM wave (S230). In this case, the output unit 130 may match an optimal change in body temperature to an optimal change in blood flow according to a height, a weight, and a stimulation area of a target person, store the matching result, and derive information on the EM wave, which most closely resembles the changes in body temperature and blood flow, as information on an optimal EM wave.

Specifically, the output unit 130 may output the optimal EM wave stimulation condition from a human body response of at least one EM wave stimulation based on a purpose of the EM wave generated by the input unit 110, a stimulation area, and information on a target person. In this case, the output unit 130 may match the optimal change in body temperature to the optimal change in blood flow according to a height, a weight, and a stimulation area of the target person, and the purpose of an EM wave and store the matching result in a database (not illustrated).

According to an embodiment of the present disclosure, a guideline may be provided to perform precise treatment by generating and applying an optimal EM wave in consideration of a target person's information.

The optimal EM wave may be generated by performing a simulation using information on a target person and information on an EM wave and by using the information on the optimal EM wave according to the purpose of rehabilitation, healthcare, or exercise prescription.

The present disclosure is described with reference to the embodiments illustrated in the drawings, but the embodiments are merely examples, and those with common knowledge in the field to which the relevant technology belongs will understand that various modifications and equivalent other embodiments may be derived therefrom. Therefore, the true technical protection scope of the present disclosure should be determined by the technical idea of following patent claims.

### DESCRIPTION OF SYMBOLS

100: electromagnetic wave stimulation optimization system
110: input unit
120: simulation unit
130: output unit

1. An electromagnetic (EM) wave stimulation optimization system comprising:
an input unit configured to receive information on a target person and information on an EM wave;
a simulation unit configured to apply the received information on the target person and the received information on the EM wave to a previously stored human phantom to perform a simulation and configured to predict a human body response to EM wave stimulation; and
an output unit configure dot output an optimal EM wave stimulation condition from the human body response of the EM wave stimulation for the information on the at least one EM wave.

2. The electromagnetic wave stimulation optimization system of claim 1, wherein
the input unit sets a stimulation area and a purpose of the EM wave based on the received information on the target person.

3. The electromagnetic wave stimulation optimization system of claim 1, wherein
the information on the target person includes an age, a height, a weight, gender, a medical history, a symptom, a stimulation area, and a specific position of the stimulation area of the target person, and
the information on the EM wave includes a frequency, an amplitude, stimulation time, a stimulation pulse, and a stimulation pulse pattern of the EM wave.

4. The electromagnetic wave stimulation optimization system of claim 1, wherein
the simulation unit applies the received information on the target person and the received information on the EM wave to a pre-stored human body phantom to perform a human body response simulation based on a Finite-difference time-domain (FDTD) and predicts a change in local body temperature of the stimulation area and a change in local blood flow of the stimulation area.

5. The electromagnetic wave stimulation optimization system of claim 1, wherein
the output unit matches an optimal body temperature change to an optimal blood flow change according to a height, a weight, and a stimulation area of the target person and use of the EM wave, stores a matching result in advance, and derives information on an EM wave causing a body temperature change and a blood flow change that are respectively most similar to the change in body temperature and the change in blood flow as information on an optimal EM wave.

6. An electromagnetic wave stimulation optimization method performed by an electromagnetic wave stimulation optimization system, the electromagnetic wave stimulation optimization method comprising:
receiving information on a target person and information on an EM wave;
applying the received information on the target person and the received information on the EM wave to a previously stored human phantom to perform a simulation and predicting a human body response to an EM wave stimulation; and
outputting an optimal EM wave stimulation condition from the human body response of the EM wave stimulation for the information on the EM wave.

7. The electromagnetic wave stimulation optimization method of claim 6, wherein,
in the receiving, a stimulation area and a purpose of the EM wave are set based on the received information on the target person.

8. The electromagnetic wave stimulation optimization method of claim 6, wherein
the information on the target person includes an age, a height, a weight, gender, a medical history, a symptom, a stimulation area, and a specific position of the stimulation area of the target person, and
the information on the EM wave includes a frequency, an amplitude, stimulation time, a stimulation pulse, and a stimulation pulse pattern of the EM wave.

9. The electromagnetic wave stimulation optimization method of claim 6, wherein,
in the predicting, the received information on the target person and the received information on the EM wave are applied to a pre-stored human body phantom to perform a human body response simulation based on a FDTD to predict a change in local body temperature of the stimulation area and a change in local blood flow of the stimulation area.

10. The electromagnetic wave stimulation optimization method of claim 6, wherein,
in the outputting, an optimal body temperature change is matched to an optimal blood flow change according to a height, a weight, and a stimulation area of the target person and use of the EM wave, stores a matching result in advance, and information on an EM wave causing a body temperature change and a blood flow change that are respectively most similar to the change in body temperature and the change in blood flow is derived as information on an optimal EM wave.
